(19) United States
(12) Patent Application Publication
(10) Pub. No.: US 2006/0068003 A1
(43) Pub. Date: Mar. 30, 2006

(54) System for increasing compliance with medication regime

(76) Inventor: Rachel S Herz, Warwick, RI (US)

Correspondence Address: Rohm & Monsanto, 12 Rathbone Place, Grosse Pointe, MI, 48230, US

(21) Appl. No.: 10/534,280

(22) PCT Filed: Nov. 6, 2003
(86) PCT No.: PCT/US03/35356

§ 371 Date: May 6, 2005

Related U.S. Application Data
(60) Provisional application No. 60/424,525, filed on Nov. 6, 2002.

(51) Int. Cl. A61K 9/20

(52) U.S. Cl. 424/464

(57) ABSTRACT

A drug-dispensing arrangement for dissociating side effects, particularly gastrointestinal side effects, such as nausea and/or vomiting, from oral ingestion of a medication eliminates the development of learned taste-aversion. If a large, random and unpredictable order of flavors, and preferably well-known and pleasant flavors, are experienced by a patient in conjunction with ingesting a specific medication, the ability to form an explicit association between any one flavor and the untoward side effect will not be possible. As an example, a supply of medication is provided to the patient in a blister pack containing a random distribution of uniformly colored pills of multiple, familiar flavors so that a patient is not able to learn to any association between the flavor/odor/color of a pill and any subsequent nausea or stomach upset. In this example, the pills can be distinctively flavored by application of a flavored coating, such as a pharmaceutical glaze, or an enteric or reverse enteric coating.

### RELATIONSHIP TO OTHER APPLICATION

This application claims the benefit of U.S. Ser. No. 60/424,525 filed on Nov. 6, 2002, the disclosure of which is incorporated herein by reference.

### BACKGROUND OF THE INVENTION

1. Field of the Invention

This invention relates generally to a system for increasing patient compliance with a medication regime, and more particularly, to a system for dissociating side effects, particularly gastrointestinal discomfort, such as nausea and/or vomiting, from oral ingestion or, in some embodiments, parenteral administration of the medication.

2. Description of the Related Art

Lack of compliance with prescribed drug regimens is a leading source of variability in drug response. Numerous studies have concluded that medication non-compliance compromises the effectiveness of therapy and is a significant contributor to the costs of medical care in every therapeutic area. Non-compliance also compromises the results of clinical studies and trials. In the case of antibiotics, the consequences of non-compliance can result in the development of antibiotic-resistant strains of bacteria.

Of all of the reasons set forth for medication non-compliance, drug side effects rank at the top of the list. While headache and nausea are the most common side effects of drug therapies, nausea has a much greater likelihood of compromising medication adherence. In fact, studies have implicated nausea as the leading cause of medication non-compliance in numerous disorders including hypertension, AIDS, depressions, cancer, and alcoholism. The debilitating consequences of nausea can be explained by the principles of learned taste-aversion.

Learned taste-aversion occurs when nausea and/or vomiting is experienced within a twelve hour period post-consumption of a substance with a specific flavor or smell. The result is that subsequent exposure to the mere smell of the substance elicits a strong sensation of nausea and an avoidance of the substance. Learned taste-aversion reflects a specific kind of conditioning where an originally neutral or positive smell-taste stimulus becomes paired with the aversive physical response of nausea, and then comes to elicit the nausea response itself. However, the phenomena is particularly potent when the flavor is novel, as is the case with most medicines which have a taste and/or smell that does not occur outside of the experience of medical intake.

Learned taste-aversions have been well-documented in the literature with respect to animals. In a typical experiment, a laboratory rat would be given a novel, pleasantly-flavored sweet drink and then made to feel sick by the injection of lithium or exposure to radiation. As a result of the experience of illness after the novel taste, the animal acquires a specific aversion for the novel taste that it was exposed to prior to the onset of illness.

Learned taste-aversions are as easily formed in humans as in rats. Most people have had the experience of eating something, getting sick soon thereafter, and then being repelled by the smell and/or taste of the food eaten prior to the illness. The effect is particularly extreme and long-lasting when the ingested food is novel, such as when sickness occurs after trying a new ethnic cuisine. Novelty is an important factor because the ingested food has no prior history of associations, so that the first association made to the novel stimulus is the one that is indelibly ingrained. Several studies have shown that the first association learned to a smell is extremely difficult to unlearn and also interferes with forming any new associations to that same odor.

Extrapolating these results to medication, when a drug with a novel flavor is consumed and nausea results as a side effect, an association will be formed readily and last durably. Conversely, pleasant, familiar flavors are more resistant to becoming associated to negative side effects because of their prior positive history. There is, therefore, a need for a system for dissociating side effects, particularly gastrointestinal discomfort, such as nausea and/or vomiting, from oral ingestion of medicine, and particularly medicine having an unusual or bad taste.

Although the pharmaceutical industry has long sought to mask or otherwise obscure the unpalatable taste, odor, and/or mouth-feel of pharmaceutical compositions, these efforts have been directed to making the individual dosage unit more palatable to increase patient compliance or, in some cases, to prevent an emetic effect. Of course, providing a palatable product is useful for maintaining customer loyalty and goodwill toward the product. However, to the best of our knowledge, there is no example in the art of a system or dispensing arrangement, for a treatment regimen, that dissociates flavor/smell from untoward side effects, particularly gastrointestinal side effects, in order to avoid learned taste-aversion.

One of the first recognized examples of learned taste-aversion was in chemotherapy treatment for cancer. A major side effect of chemotherapy is nausea. It has been shown in a number of studies that what a patient eats before a chemotherapy session will later elicit a learned taste-aversion response. It has also been shown that novelty is a factor in this situation. The more familiar and bland the food eaten prior to chemotherapy, the less likely it is to trigger a learned taste-aversion response in the future. These studies, however, were not directed to the impact of nausea on the patient's subsequent reaction to the chemotherapeutic agent, but rather to the food ingested prior to chemotherapy, and to food in general (anorexia).

Although non-compliance in chemotherapy patients is a serious concern, these patients are usually more willing to tolerate nausea side effects due to the gravity of the disease than patients suffering from less imminently life-threatening illnesses. Nevertheless, there is a need for making the administration of chemotherapy a less onerous burden to the patient.

Learned taste-aversion is particularly a problem for medication that is prescribed in a continuous treatment regime. In this case, the patient will associate the odor or taste of the medication with the effect of the medication. Some patients will become nauseated before ingestion of the medication. Often, even the mere smell of the medication released from opening a medicine bottle can cause the patient to make a decision not to take the medication. There is, therefore, a need for a system, or dispensing arrangement, for increasing patient compliance with a prescribed drug regimen by avoiding learned taste-aversion.

It should be noted that taste-aversion is particularly a problem with children (and the elderly). Since, the Food and Drug Administration (FDA) now requires pediatric formularies for all medicines that are prescribed for adults, there is a need for child-friendly medication that avoids the development of learned taste-aversion.

Various techniques have been proposed to mask taste and/or odor, including highly compressed tablets or pills so that they will not disintegrate in the mouth; providing taste-masking coatings; providing the medication, in liquid or solid form, inside an un-flavored gelatin capsule; providing the active agent in a flavored, liquid suspension; adding taste inhibitors, taste maskers, flavorings and flavor augmentors; forming salts that have less taste; forming adsorbates with synthetic cationic exchange resins; concealing the drug in a food matrix; etc. In addition to the foregoing, compositions have been proposed that include ingredients to inhibit or prevent undesirable side effects of the type that would cause non-compliance. There is a need, however, for a simple, inexpensive system, or drug dispensing arrangement, that obviates the need for complicated manufacturing techniques and the addition of expensive ingredients. Of course, there remains a need for a system, or drug dispensing arrangement, that clearly dissociates side effects, particularly gastrointestinal discomfort, such as nausea and/or vomiting, from oral ingestion of medication.

It is, therefore, an object of this invention to provide a system for dissociating side effects, particularly gastrointestinal discomfort, such as nausea and/or vomiting, from oral ingestion of medicine, and particularly medicine having an unusual or bad taste.

It is also an object of this invention to provide a dispensing arrangement for a drug treatment regimen that dissociates negative side effects from oral ingestion of medicine, and thereby increases patient compliance and comfort.

### SUMMARY OF THE INVENTION

The foregoing and other objects, features, and advantages are accomplished by this invention which is a system for dissociating side effects, particularly gastrointestinal side effects, such as nausea and/or vomiting, from administration of a medication, and particularly from oral ingestion of a medication, in order to eliminate the development of learned taste-aversion. In accordance the invention, the connection between the smell/taste of the drug and any discomfort caused by ingestion of the medication, is preferably accomplished without changing or altering the active drug component. In a practical embodiment of the invention, a drug dispensing arrangement is provided to counteract the development of taste-aversion.

In order to counteract the conditioning principles involved in learned taste-aversions, it is important that medicines are created with flavors that are well-liked and known outside of, and prior to, the medical experience. In addition, it is important that the explicit association between a particularly flavored substance and a response outcome, such as nausea, is dissociated. In accordance with the principles of the invention, if a large, random and unpredictable order of flavors, and preferably well-known and pleasant flavors, are experienced in conjunction with one specific medication, the ability to form an explicit association between any one flavor and the untoward side effect will not be possible.

Further, most medications that cause nausea as a side effect, do not do so every time. Thus, if many different, random flavors are provided, sometimes one of those flavors may be associated with nausea, but at other times it would not. These sporadic connections further ensure that a generalized conditioned response of nausea to the medication, as a whole, would be unlikely to form. Of course, a sufficient number of positive, or pleasant, flavors must be provided to assure that associations are not formed. In an illustrative embodiment of the invention, at least ten flavors are provided.

In an illustrative embodiment of the invention, a supply of medication is provided to the patient in a flavor-dissociating drug dispensing arrangement, illustratively, a blister pack containing a random distribution of uniformly colored pills of multiple, familiar flavors so that a patient is not able to learn to any association between the flavor/odor/color of a pill and any subsequent nausea or stomach upset.

As used herein, the term “drug dispensing arrangement” is used to refer to a means for distributing, or dispensing, a course of medication in a prescribed treatment regimen, or as part of a treatment regimen that can be short-term or long-term. This would include a means for dispensing the individual dosage unit(s) in a prescription, which for example, might be 90 tablets for a 30 day supply of an individual dose that is meant to be taken three time daily.

The individual dosage units, or drug depots, are preferably in the form of pills, tablets, caplets, capsules, lozenges, and the like, comprising the active ingredient(s) and physiologically-acceptable fillers and excipients as are known in the art. The active ingredients may be any therapeutic, diagnostic, or otherwise bioactive agent or combination of agents. Physiologically-acceptable fillers and excipients include, without limitation, auxiliaries, such as granulating and disintegrating agents, e.g., starch; binders, such as polyvinyl pyrrolidone; lubricating agents, such as magnesium stearate or talc; pH adjusting agents; bulking agents, such as microcrystalline cellulose, etc. The ingredients may be combined according to well-known techniques, such as dry or wet granulation followed by compression into a shaped dosage form.

In a preferred embodiment, the individual dosage units are distinctively flavored by application of a flavored coating, such as a pharmaceutical glaze, or an enteric or reverse enteric coating. Techniques for applying coatings are well-known in the art. Even though the pills would be swallowed, rather than chewed, the brief exposure to the flavor would have prior positive associations. Of course other means of flavoring can be employed, such as by providing the active ingredient in a flavored solid or liquid matrix of inert ingredients, illustratively, as an oral suspension or in a food matrix, such as a cookie, cake, cracker, hard or soft/chewable candy, or the like.

In a specific illustrative embodiment, a solid dosage unit, illustratively a pill, is provided with a flavored thin, quick-dissolving polymeric coating, such as an enteric coating or a reverse enteric coating as known in the art. U.S. Pat. Nos. 5,489,436; 5,084,279; or 5,215,755, the disclosures of which are incorporated herein by reference, teach suitable coatings and methods of making and applying same. Preferably the polymer coating is water soluble so that the flavor is encountered during the brief exposure to the mouth. Illustrative examples include, without limitation, synthetic or semi-synthetic polymeric coating materials, such as hydroxypropyl methylcellulose, such as the Pharmacoat brands available from Shinetsu, Tokyo, Japan; methylcellulose, such as the Methocel brands available from Dow Chemical, Midland, Mich.; polyvinyl alcohol; polyvinyl acetate; cellulose acetate butyrate; styrene acrylate copolymers; or copolymers of acrylic acid esters, such as the Eudragit brands from Rohm Pharma, Germany.

Natural and synthetic flavoring agents for polymer coating materials are well-known. The flavors are preferably pleasant, and most preferably familiar flavors, such as peppermint, spearmint, chocolate, vanilla, butterscotch, or fruit flavors, such as orange, lemon, strawberry, or raspberry. The foregoing list is merely illustrative and is not intended to be limiting in any way.

In addition to the foregoing, the color of the pills is preferably uniform. Uniform coloring ensures that the patient is not able to form an association to guess the flavor, or the sequence of flavors in a multiple pill arrangement. However, random coloring and/or coloring of the pills with a color that is typically not associated with the flavor of the pill, would also work provided that the system of coloring/flavoring results in an inability of the patient to form an explicit association between a particular colored/flavored pill and a response outcome, such as nausea.

Alternatively, the dosage unit may be in the form of a liquid, such as an oral suspension or emulsion. In this embodiment, the active ingredient(s) are suspended or dissolved in a liquid, such as water or alcohol, with the flavoring agent(s) and other auxiliary agents, including, without limitation, preservatives, stabilizers, wetting or emulsifying agents, coloring agents, and natural or artificial sweeteners. Techniques for making oral suspensions, solutions, or emulsions are well-known in the art.

The technique used for flavoring and/or coloring the individual drug depots should not interfere with the pharmacokinetics of drug release so that the drug's bioavailability will not be modified or impaired by the implementation of the invention. Of course, the flavoring/coloring must also comply with any required governmental regulations, such as FDA regulations, in terms of efficacy and safeness and coloration requirements.

In the specific illustrative embodiment of the invention described herein, the pills are dispensed in a blister pack. The blister pack prevents the smells of each pill from intermingling so that a collective “medicinal smell” or an unfamiliar or unidentifiable smell cannot form, smell being the most important aspect in distinguishing flavors. It should be noted that flavor is a composite of smell and the five basic tastes (salt, sour, sweet, bitter, and umami (savory)). For example, the flavor of peppermint is due to the smell of peppermint plus the taste of sweet.

Of course, other dispensing arrangements can be devised in accordance with the principles of the invention. Illustratively, individual doses of the drug in a prescribed treatment regimen can be flavored and provided to the patient in individual wrappings, or individual doses can be provided, in liquid form, in individual vials or ampules having a variety of flavors (and preferably uniform color) so that the flavor that cannot be detected or predicted prior to ingestion.

Many medications are delivered parenterally, i.e., outside of the digestive system, such as by intravenous administration, or intramuscular, subcutaneous, or intraperitoneal injection. Chemotherapy, in particular, is typically delivered by intravenous administration. Alternative embodiments of the invention may be required to address drugs that are administered parenterally.

Although odors are not as strongly associated with a nausea response as oral ingestion of a substance, it is possible that by accompanying an intravenous (IV) chemotherapy treatment with a novel odor, or scent, the patient will associate his sickness with the smell of the odor, rather than his last meal, particularly if the odor is the last thing experienced by the patient. In this embodiment, a distinctive odor of a type that would not ordinarily be encountered, would accompany the IV treatment(s). Alternatively, an odor from a set, or array, of distinctive odors, could accompany multiple, sequential treatments in a random manner.

Illustrative techniques for providing an odor in conjunction with IV therapy include the use of fragrance dispensers of the type used for room freshening and fragrance-soaked wipes. The fragrance-soaked wipes could incorporate the chemical substance used to produce the novel odor in a cotton or cellulosic matrix, and, in some embodiments, alcohol or other disinfectant so that the wipe can be used to clean the area where the IV is to be inserted.

Listed below are exemplary chemicals that could be used in certain specific illustrative embodiments of the invention to accompany IV treatment with an odor. They are all unfamiliar and with a very low likelihood of being encountered in daily life but they are not inherently unpleasant. The following chemicals can be obtained commercially, for example, from International Fragrance and Flavors, Union, N.J. The hedonic perception may vary with concentration. The following concentrations, in diethyl phthalate, an odorless organic solvent, results in hedonic (or pleasantness) values that vary by odor but overall are in the neutral to slightly unpleasant range as baseline ratings.

| Chemical Name | Concentration (%) |
|---|---|
| Ditmetol | 10 |
| Phenoxanol | pure |
| Amyl salycilate | pure |
| Agrumea | 10 |
| Orange flower ether | 2.5 |
| iso butyl quinoline | 10 |
| intrelever ald | 10 |
| Ylang | pure |
| hexenol | 10 |

In still other embodiments of the invention, IV therapy can be accompanied by a pleasant odor in order to evoke a positive emotion or mood, or a feeling of well-being. Novel odors are particularly susceptible to forming emotional associations and as a function of the connected emotional association, hedonic response to odors can be directly changed and long-lasting. In this manner, a patient may be less predisposed to being uncomfortable both during and after treatment.

In another embodiment, the IV treatment can be accompanied by the administration of a flavored oral distractor, i.e., a placebo, flavored/colored and/or dispensed in accordance with the principles of the present invention, so that the patient attributes any sickness response(s) to the oral distractor rather than the IV treatment.

### BRIEF DESCRIPTION OF THE DRAWING

Comprehension of the invention is facilitated by reading the following detailed description, in conjunction with the annexed drawing, in which:

FIG. 1 is a schematic representation of a flavor-dissociating dispensing arrangement for medication, in the form of pills, in accordance with the present invention.

### DETAILED DESCRIPTION OF THE INVENTION

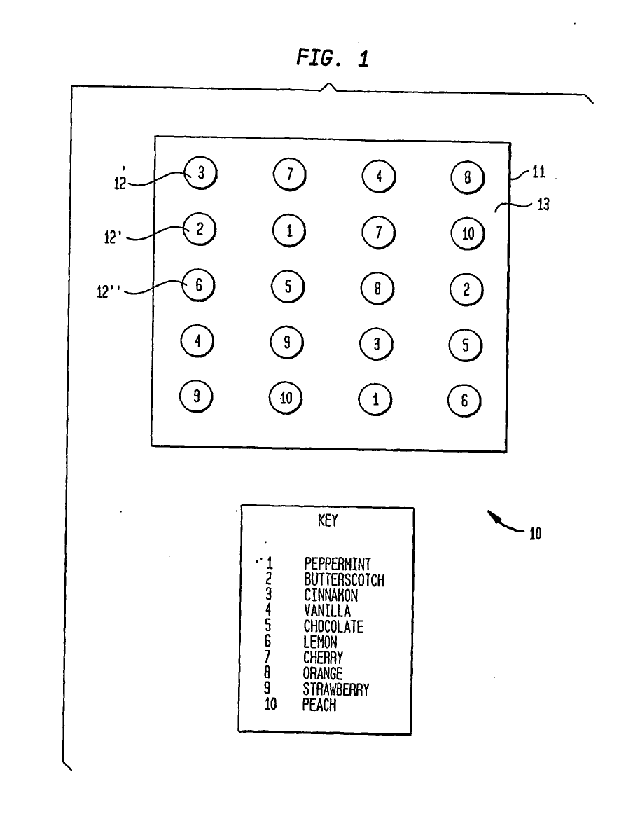

FIG. 1 is a schematic representation of an illustrative flavor-dissociating dispensing arrangement in accordance with the present invention. The arrangement is in the form a blister pack 10 having a foil backing or lower substrate 11 on which a plurality of individual dosage units of a medication, in the form of pills 12, 12′, 12″, etc., are disposed, illustratively, in a 4×5 grid pattern as shown in FIG. 1. A protective plastic layer 13 covers the pills forming, in conjunction with the backing 11, a protective seal against external elements and isolating the pills from each other by being adhesively bound to foil backing 11 in those areas not occupied by pills. The individual dosage units are dispensed by applying sufficient pressure to plastic layer 13 to rupture foil backing 11.

In this particular embodiment, pills are provided with a coating having one of ten distinctive flavors. The flavored pills are arranged randomly in blister pack 10, illustratively as shown in FIG. 1, where pills 12 have the flavors labeled in accordance with the inset key: peppermint 1, butterscotch 2, cinnamon 3, vanilla 4, chocolate 5, lemon 6, cherry 7, orange 8, strawberry 9, and peach 10. Of course, these flavors are merely exemplary and are not intended to be limiting. The only criteria are that the flavors are pleasant and familiar. As noted above, the color of the individual pills 12, 12′, 12″, etc., is preferably uniform so that the patient cannot guess or predict the flavor or sequence of flavors in the arrangement.

It is believed that the principles of the invention would have widespread application in the treatment of diseases, or other conditions, in which learned taste- or odor-aversion to an administered therapeutic, diagnostic, or otherwise bioactive agent or agents impacts patient reaction to the administered agent(s).

The immune system is involved in mobilizing physiological defenses against foreign substances that enter the body so that these substances do not cause disease. The immune system, however, is also subject to neural control. In other words, how you think can affect how your immune system functions. Thus, the immune system can be conditioned. In fact, research on conditioned modifications of the immune system developed from work on taste-aversion conditioning. One drug that is particularly effective in producing conditioned taste aversions in rats is cyclophosphamide. An important physiological effect of cyclophosphamide is that it suppresses the immune system. That is, rats who are given cyclophosphamide are prone to diseases because cyclophosphamide interferes with the immune system's production of antibodies. It has been experimentally shown that suppression of the immune system can also be elicited as a conditioned response to a taste previously associated with cyclophosphamide. For example, in one experiment, rats drank a novel saccharin solution and then were injected with cyclophosphamide. Several days later the animals were injected with a foreign tissue and the production of antibodies was measured in two groups of rats. One group of rats was then re-exposed to the saccharine solution that had been associated to cyclophosphamide and another group was not. It was found that the group that was re-exposed to the saccharine solution showed a suppression in their immune response and did not produce antibodies to combat the foreign tissue, while those who were not re-exposed to the saccharine solution exhibited a normal immune response. This shows that the flavor associated to a drug that has effects on the immune system can subsequently elicit those effects by itself.

While the experimental examples have been negative, that is, demonstrate immune suppression, it is reasonable to expect the converse since the immune system is subject to neural control. Therefore, pairing a drug that enhances immune system response with a novel flavor and then presenting the novel flavor alone should elicit enhanced immune system response. This mechanism of flavor-conditioned immune enhancement would be particularly relevant to cancer treatment, as the individual's immune system is strongly implicated in how well they will respond to treatment and recovery. Therefore, the application of the principles of the present invention, to the administration of medications that impact the immune system, either alone or in combination with other therapeutic agents, would have the additional benefit of improving, not only patient compliance and comfort, but also immune system response.

In addition to the foregoing, the invention may be useful in counteracting conditioned tolerance to opiate pain killers. The most serious and common side effect of administration of opiate pain medication is the conditioned tolerance that develops from continued use. Conditioned tolerance to opiates occurs when a dose that was originally appropriate in managing pain is no longer effective and an increasingly higher dose is required in order for an acceptable level of pain reduction to be felt. The most serious consequence of high levels of opiate usage is that it can lead to post-treatment addiction. Conditioned tolerance to opiates occurs because the cues that surround drug intake become associated with the drug's physical effect and as a result elicit a counteracting physiological response. One of the main anticipatory cues of drug intake is the drug's flavor and smell. If the drug's flavor and smell were dissociated from the drug, following the principles of the present invention, conditioned tolerance would be reduced and the patient would require less drug to achieve the desired effect. This would, in turn, reduce the risk of addiction.

Of course, the principles of the invention can be applied to dispensing arrangements used for pharmaceuticals in clinical trials to prevent attrition of participants as a result of learned taste-aversion, and consequently, inconclusive data.

In addition to the foregoing, the principles of the invention may also find application to products other than medicinal products, illustratively household products, such as cleaning solutions, which may repel a user by evoking an emotional and/or physiological response through their smell.

Although the invention has been described in terms of specific embodiments and applications, persons skilled in the art may, in light of this teaching, generate additional embodiments without exceeding the scope or departing from the spirit of the claimed invention. Accordingly, it is to be understood that the drawing and description in this disclosure are proffered to facilitate comprehension of the invention and should not be construed to limit the scope thereof.

what is claimed is:

1. A drug dispensing arrangement for a medication for dissociating negative side effects from intake of the medication comprising administering multiple individual dosage units of the medication in a treatment regimen in a manner that prevents the development of taste- and/or odor-aversion by preventing a patient from forming an explicit association between a particular color/flavor/odor of the medication and a negative response outcome.

2. The drug dispensing arrangement of claim 1 wherein the step of administering comprises providing the medication in multiple individual dosage units having associated therewith a plurality of distinctive flavors and/or odors, the individual dosage units being administered so that the flavors and/or odors are presented to the patient in a random and unpredictable order.

3. The drug dispensing arrangement of claim 2 wherein the individual dosage units are in the form of solid drug depots.

4. The drug dispensing arrangement of claim 3 wherein the solid drug depots have flavored coatings.

5. The drug dispensing arrangement of claim 4 wherein the flavors are familiar and pleasant.

6. The drug dispensing arrangement of claim 4 wherein the coatings on the individual dosage units have a uniform color.

7. The drug dispensing arrangement of claim 4 wherein the coatings on the individual dosage units in the treatment regimen are provided in a random distribution of multiple colors.

8. The drug dispensing arrangement of claim 4 wherein the coatings on the individual dosage units have a color that is not typically associated with the flavor of the coating.

9. The drug dispensing arrangement of claim 2 wherein the plurality of flavors comprises at least ten (10) flavors.

10. The drug dispensing arrangement of claim 2 wherein the individual dosage units are in liquid form.

11. The drug dispensing arrangement of claim 2 wherein the individual dosage units are in the form of a food matrix.

12. The drug dispensing arrangement of claim 1 wherein the step of administering comprises parenteral administration of the medication.

13. The drug dispensing arrangement of claim 12 wherein parenteral administration is accompanied by the provision of a distinctive novel odor.

14. The drug dispensing arrangement of claim 13 wherein the distinctive novel odor is unfamiliar yet not unpleasant.

15. The drug dispensing arrangement of claim 11 wherein individual doses in a treatment regiment of multiple, sequential parenteral administrations of a medication are accompanied by plurality of distinctive novel odors in a random pattern.

16. The drug dispensing arrangement of claim 13 wherein the odor is delivered from a fragrance dispenser.

17. The drug dispensing arrangement of claim 13 wherein the odor is delivered from a fragrance-soaked wipe.

18. The drug dispensing arrangement of claim 12 wherein parenteral administration is accompanied by the provision of a flavored oral distractor.

19. A dispensing arrangement for medication comprising:
a foil backing;
a plurality of individual dosage units of a medication in the form of pills disposed on the foil backing, the plurality of pills having individually one of multiple flavors and being arranged on the foil backing so that the flavors occur in a random pattern; and
a protective plastic layer covering the pills and adhesively bound to the foil backing in the areas not occupied by pills.

20. The dispensing arrangement of claim 19 wherein each of the multiple flavors are familiar and pleasant.

21. The dispensing arrangement of claim 19 wherein the flavor is imparted by a coating on the individual dosage units.

22. The dispensing arrangement of claim 21 the coatings on the individual dosage units have a uniform color.

23. The dispensing arrangement of claim 19 wherein the multiple flavors comprises at least ten (10) flavors.
